(19) United States
(12) Patent Application Publication
(10) Pub. No.: US 2005/0165298 A1
(43) Pub. Date: Jul. 28, 2005

(54) Treatment of cardiac tissue following myocardial infarction utilizing high intensity focused ultrasound

(75) Inventors: Eugene A. Larson, Lummi Island, WA (US); Perry W. Kaminski, Stehekin, WA (US)

Correspondence Address: ROBERT L. MCDOWELL, 1170 JACKSON HEIGHTS DR, WEBSTER, NY, 14580-9367, US

(73) Assignee: Crum, Kaminski & Larson, LLC

(21) Appl. No.: 11/084,568

(22) Filed: Mar. 18, 2005

Related U.S. Application Data
(63) Continuation-in-part of application No. 10/921,715, filed on Aug. 19, 2004.
(60) Provisional application No. 60/500,067, filed on Sep. 4, 2003.
(60) Provisional application No. 60/560,089, filed on Apr. 7, 2004.

(51) Int. Cl. A61B 5/05

(52) U.S. Cl. 600/410

(57) ABSTRACT

A noninvasive or minimally invasive treatment of infarct areas of the heart with High Intensity Focused Ultrasound (HIFU) emitted without respect to the timing or phase of the cardiac cycle, intended to remodel cardiac tissue by inducing angiogenesis and/or the formation of myocytes to improve cardiac function.

This application is a Continuation-in-Part of application Ser. No. 10/921,715 filed Aug. 19, 2004 which claims the benefit of U.S. Provisional Patent Application No. 60/560,089 filed Apr. 7, 2004 and U.S. Provisional Patent Application No. 60/500,067 filed Sep. 4, 2003.

### FIELD OF THE INVENTION

The present invention is directed to the noninvasive or minimally invasive stimulation of cardiac myocardial revascularization and/or myocardial tissue regeneration.

### BACKGROUND OF THE INVENTION

In the United States, cardiovascular disease results in over five million hospitalizations and approximately one million deaths each year. Approximately 70 million Americans are afflicted with one or more types of cardiovascular disease. Approximately 7.1 million Americans are afflicted with a Myocardial Infarction (heart attack) annually and approximately five million develop congestive heart failure.

Myocardial Infarction (from sudden localized areas of ischemia due to emboli or thrombi) results in areas of necrosis in an area of the heart muscle.

The necrosis results in reduced thickness of the cardiac wall as the necrotic muscle is removed by mononuclear cells. The infarcted area gradually converts into a shrunken thin scar, beginning at the periphery of the infarct and gradually moving centrally. The necrotic area may also expand as cells on the periphery are damaged by increased stress during contraction of the heart wall.

Many complications, some immediately fatal, develop from the infarct including ventricular arrhythmias, reduced cardiac output and heart failure in which the heart is unable to pump blood at a rate commensurate with the requirements of the metabolizing tissues.

There is currently no accepted therapy to cause revascularization and/or regeneration of myocardial cells and tissue in an infarct region of the heart. Research has indicated that sublethal injury to muscle tissue induces repair and remodeling of scar tissue combined with the formation of blood vessels (angiogenesis). Various methods of experimental introduction of energy to injure or stimulate myocardial tissue include laser tissue perforation and acoustic shock wave. Stem cell and myocyte implantation are being explored as a means of inducing cardiac tissue remodeling.

A method of treating cardiovascular conditions is disclosed in U.S. Pat. No. 5,817,021 to Reichenberger wherein therapeutic ultrasound is delivered to a desired region of the heart with an intensity such that tissue modifications (e.g. necrotization) are produced by the thermal effect of the ultrasound waves in the targeted tissue area. In the disclosed method, delivery of the therapeutic ultrasound is required to be synchronized with the heart activity. Therapeutic ultrasound is emitted only during such phases of heart activity wherein the heart and vessels are at relative mechanical rest (e.g. diastole). Thus, therapeutic ultrasound is delivered in an interrupted partial cardiac cycle manner and therefore ultrasound waves required for achieving a therapeutic effect are present only during the emission which occurs while the heart is at rest. However, targeting only during diastole results in the inability to achieve a thermal dose throughout the region of interest (ROI) to induce modifications.

### SUMMARY OF THE INVENTION

The present invention is directed to the noninvasive or minimally invasive treatment of cardiac ischemia and infarction by treating the tissue with heat produced by emission of ultrasound (including High Intensity Focused Ultrasound or HIFU) in a continual manner throughout, and without respect to the timing of the heart cycle, to have a biological and/or therapeutic effect, so as to remodel the tissue in the ischemic or infarcted area.

### BRIEF DESCRIPTION OF THE DRAWINGS

FIG. 1 shows a lesion produced intraoperatively in the posterior wall of an animal heart.

FIGS. 2A and 2B are photographs of sub-lethal damage to arterial wall tissue produced by relatively low levels of HIFU.

FIGS. 3A, 3B and 3C illustrate, respectively, linear, spherical, and sectioned annular phased arrays of ultrasound transducers.

FIGS. 4A and 4B show field distributions of, respectively, time averaged intensity and heat rate of a 20 element sectioned annular phased array.

FIGS. 5A, 5C, 5E, 5G and 51 show temperature evolution at different time intervals while FIGS. 5B, 5D, 5F, 5H and 5J show respective lesion formation due to HIFU exposure for the model shown in FIGS. 4A and 4B.

### DETAILED DESCRIPTION OF THE PREFERRED EMBODIMENT

The present invention describes the stimulation of controlled myocardial remodeling and/or angiogenesis resulting from sublethal cellular heating utilizing High Intensity Focused Ultrasound (HIFU). Ablation and cell death occurs at about 60° C. or above; structural protein remodeling, changes in the shape of protein and phase transition occur between about 50° C. and about 60° C.; and at about 40° C. or below, no permanent cellular changes or damage occurs.

An in vivo animal experiment was designed and carried out to demonstrate the effectiveness of producing a lesion using High Intensity Focused Ultrasound (HIFU) in a live pig heart. The goal was to produce a lesion in the endocardium of the posterior left ventricular wall by applying HIFU intraoperatively through the heart from the epicardial surface of the anterior left ventricular wall. The unfocused HIFU energy passed first through the anterior myocardium of the left ventricle, then through the blood-filled ventricular chamber to reach the endocardium of the posterior left ventricular wall where the HIFU power was focused. Tissue within the focal region, where the spatial peak intensity was greatest, was heated due to absorbed energy creating a lesion.

For this study, a HIFU system was utilized with total forward electrical power set to 60 watts. A HIFU transducer was selected with 4 MHz center frequency and a 5 cm fixed focal length. Because the region of interest in the myocardium was less than 5 cm from the front face of the transducer a truncated hydrogel cone was placed between the transducer and the epicardium to serve as an acoustic standoff. Hydrogel was chosen as the acoustic coupling path within the standoff because it is easy to handle and it is relatively unattenuating to the unfocused ultrasound energy propagating through it.

The transducer with truncated conical standoff was placed on the anterior left ventricular wall of the beating heart and continuous wave (CW) acoustic power applied in a single burst of ten seconds. Ultrasound energy generated within the transducer passed through the hydrogel, the anterior wall of the heart, the blood-filled ventricle, and focused on the endocardium of back wall of the left ventricle.

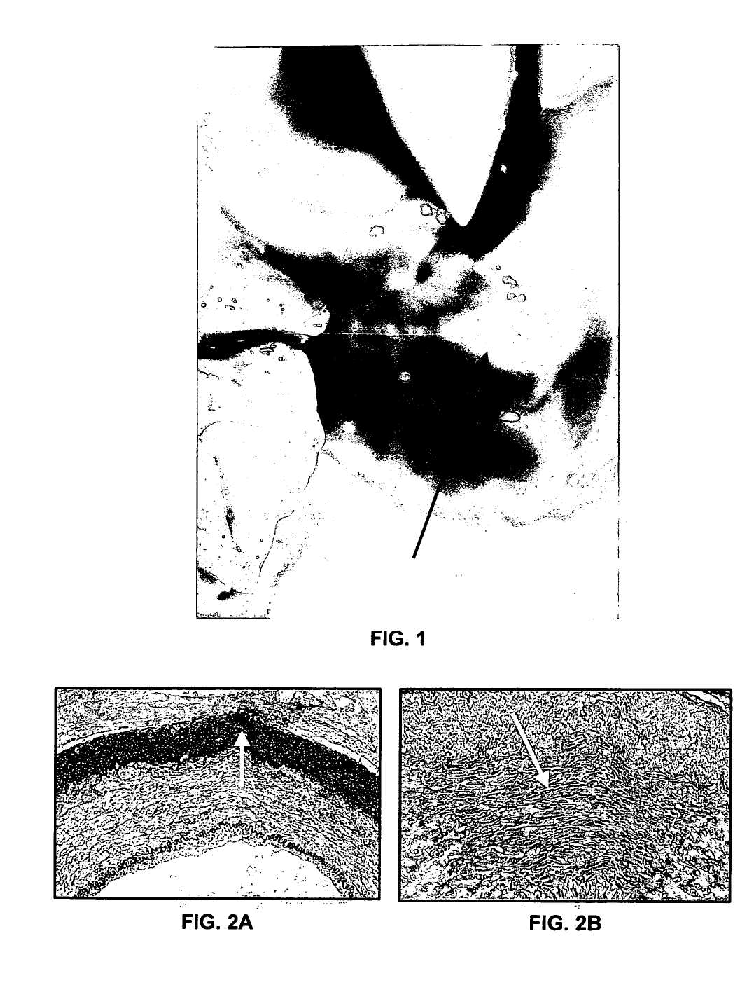

A lesion on the posterior ventricular myocardium was successfully created using HIFU applied from the anterior wall through the left ventricular cavity to the posterior wall. The photograph in FIG. 1 shows the lesion produced intraoperatively in the posterior wall with the transducer device placed on the epicardium of the anterior left ventricular wall. The transducer and the origin of the HIFU are to the right of this picture. HIFU energy passed through the anterior wall, the blood-filled ventricular chamber and focused on the endocardium of the opposite posterior left ventricular wall as indicated in this picture. Intervening tissue (the anterior wall) appeared undamaged.

FIGS. 2A and 2B are photographs of sub-lethal damage to arterial wall tissue produced by relatively low levels of HIFU. In FIG. 2A the arrow points to a layer of tissue stained by a Van Gleason stain to show elastin fibers. Note the disruption in the layer. Similarly, FIG. 2B shows tissue stained by a trichrome stain to show collagen fibers. Note the obvious disruption in the fibers. In both cases, the damage produced to these tissues is sub-lethal and will be structurally repaired by the body. It is during this structural repair that myocyte formation, tissue regeneration and remodeling and revascularization resulting from angiogenesis will occur. The arrow in FIG. 2A shows that the elastin fibers (stained black) are damaged, and disrupted. FIG. 2B shows a higher magnification of the area shown in FIG. 2A, and shows that the collagen fibers (indicated by the arrow), located distal to the elastin fibers, are also damaged, although not lethally.

The present invention provides a method for remodeling (including myocyte formation, angiogenesis and/or tissue regeneration) of cardiac tissue within the heart. The method comprises targeting a region of interest of the heart, such as with diagnostic ultrasound, fast computed tomography (CT), or Magnetic Resonance Imaging (MRI), emitting therapeutic ultrasound energy (HIFU) from an ultrasound radiating surface, focusing the emitted therapeutic ultrasound energy on the region of interest and, producing sub-lethal cellular or tissue damage in the region of interest of the heart, such as, the ventricular wall, or any other location within the heart.

Preferably, the inventive method achieves the myocardial tissue remodeling by steps which include:
(a) imaging the area of therapeutic interest of the heart and/or the attached vessels;
(b) gating the tissue/blood interface so as to allow the delivery of High Intensity Focused Ultrasound (HIFU) in a continual manner, without timing to the heart's cycle or phase, to the ROI during any phase of the cardiac cycle; and
(c) delivering ultrasound to or near the area of infarction and resulting scarring with an ultrasound device to induce a controlled amount of cellular damage and/or tissue remodeling to a localized area of the heart and/or the included blood vessels.

Most preferably, the steps of the inventive method include:

1. Imaging of the heart and specifically the area of therapeutic interest by two or three dimensional Transesophageal Echocardiography or Transthoracic Ultrasound using phased or annular array imaging.

2. Identifying and gating a structural landmark of the heart wall such as epicardial surface or the endocardium at the tissue/blood interface to dynamically focus the same or another single or multiple annular or phased array transducer (in the frequency range of 1 to 7 MHz) so as to deliver HIFU continuously to the moving interface. For example, gating of the endocardium/blood interface may be implemented as follows:
(a) The operator of the system identifies the endocardium/blood interface from a one-dimensional m-mode (selected from the imaging ultrasound array) and positions an electronic “gate” around the excursion of the heart wall in the vicinity of the ROI.
(b) The electronic imaging system (from step 1) tracks the echo within the gate window as it moves axially and generates an analog voltage depth signal.
(c) The analog depth signal drives the dynamic focus of the HIFU transducer (changes the electronic phasing to each element of the imaging array to modify the acoustic delay on the fly).
(d) Feedback may be provided to the operator by superimposing the HIFU focus on the image.

3. In the case of creating an area of sublethal injury to cells where exact acoustic path properties and location are critical, or when utilizing cell-lethal HIFU for treating the perimeter of the infarct area to destroy nerve budding and/or create a lesion to electrically block arrhythmia resulting from the infarct, a micro ultrasound device (combined transmitter and hydrophone transducer) that permits precise location of the scar tissue and intended site of the therapeutic HIFU focus at the point of scar tissue on the ultrasound image (transponder), may be utilized. The “transponder” provides an intracardiac transmit source for phase aberration correction (transmitter), and functions as a hydrophone for confirming the location of the HIFU focus before therapy is initiated.
(a) The scar tissue may be mapped during imaging or with a cardiac catheter mounted ultrasound transducer serving as a transponder. The catheter may include capability for electrically mapping areas of the heart to identify the infarction and/or resulting arrhythmias.
(b) The point-source nature of the micro catheter transducer/transponder in (a) above may be utilized with time-reversal algorithms to remove phase aberrations resulting from multiple acoustic paths. Phase aberration correction of the HIFU focus may not be necessary, such as when imaging by Transesophageal (TEE), as the tissue is more uniform than with Transthoracic echocardiography.
(c) The location of the HIFU focus prior to initiating a therapeutic power level may be confirmed by pulsing the HIFU transducer at low power, such as to have no biological effect, and locating the HIFU focus and intensity with the micro catheter transducer/transponder.
(d) The location of the HIFU focus may also be determined by the observation of hyperechogenicity at the site of the HIFU focus from the production of small microbubbles induced by the applied HIFU pulse in the tissue.

4. The directed HIFU acoustic energy is preferably varied so as to induce cellular damage or modification to selected cells within a specific localized area of the heart. The controlled introduction of cellular damage will result in partial damage to collagen and muscle fiber tissue as seen in FIG. 2A or 2B, or changes in the shape of proteins, structural protein remodeling and phase transition (temperatures of about 50° C. to about 60° C.). In either case, tissue regeneration or structural remodeling, resulting from this induced heat from ultrasound (HIFU), will occur.

The inventive method thus provides for the non-invasive or minimally invasive treatment of heart muscle damaged by ischemia resulting from Myocardial Infarction utilizing HIFU (preferably in the frequency range of 1-7 MHz, but not limited thereto), to:
(a) create a well controlled region of damage to scar and native myocardial tissue of determinable volume (depth and shape), which neither bleeds, chars nor immediately erodes, to cause the formation of new blood vessels (angiogenesis), the formation or proliferation of myocytes and/or the remodeling of tissue.
(b) Cause injury to cardiac cells, or cause phase transition, changes in the shape of cell proteins or structural protein remodeling in a well defined volume, so that they regenerate over time in a predictable manner which restores function to cardiac cell tissue which has been damaged by ischemia and/or infarction

The inventive method is preferably carried out through utilization of the following:

1. Two or three dimensional phased or annular array imaging and gating of the heart endocardium through Transesophageal or Transthoracic ultrasound imaging allows for dynamically controlling the continuous therapeutic ultrasound focus in the diseased heart whereas synchronizing to an ECG signal does not represent true heart wall and vessel motion. Transesophageal imaging with HIFU therapy is particularly applicable, given the proximal location of the esophagus to the heart.

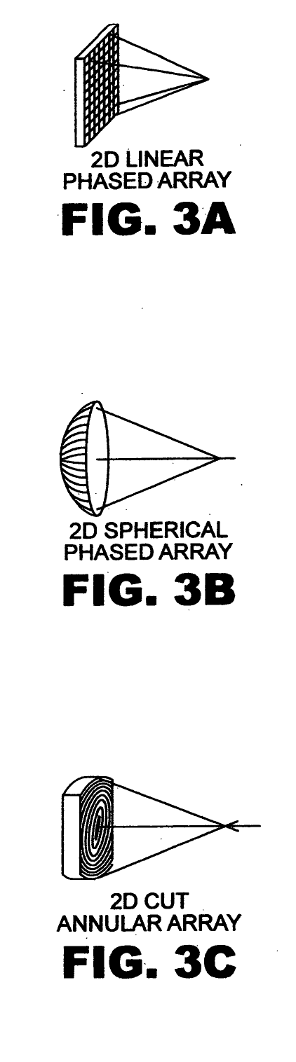

2. Array therapy HIFU transducers (single or multiple) dynamically focused by a gated signal from ultrasound imaging, as in 1 above. The transducer may be an annular, oval or linear phased array (as depicted in FIGS. 3A, 3B and 3C) in the frequency range of 1-7 MHz. The HIFU therapy transducer can be the same transducer that is used for ultrasound imaging or a separate transducer used in synchrony with ultrasound imaging or other imaging modality, such as MRI.

3. In the case of creating a region of treated cells where exact acoustic path properties and location are critical, an in-dwelling catheter based cardiac acoustic transponder/hydrophone/transmitter can be utilized. A thin film plastic or ceramic piezoelectric chip mounted on a catheter lead which:
(a) permits location of HIFU transducer focus as well as the therapy area on the ultrasound image.
(b) provides a point source ultrasound transmitter from the site of therapy interest back to both the HIFU and the imaging transducer which in turn provides phase aberration correction feedback data for accurately generating the HIFU focus and provides a method for overcoming diffraction limits by expanding the effective aperture of the ultrasound transmitter.
(c) provides a direct measure of tissue attenuation in the desired path so that accurate assessments of the acoustic intensities generated by the source transducer that will be required to induce a desired biological effect.

4. The design of a transducer array can take many forms. Some specific approaches to this array design as well as some details on the use of this array to produce either lethal or sub-lethal effects in cardiac tissue are provided below.

The following HIFU system design can be utilized for either Trans-esophageal or Trans-thoracic treatment of heart tissue to introduce remodeling and/or revascularization. In one embodiment, the system is composed of two-dimensional, multi-channel-multi-element arrays that will be used in both imaging (low power, high dynamic range) and treatment (high power, low dynamic range) modalities. The ultrasound transducers can be linear, spherical, or sectioned annular phased arrays (as shown in FIGS. 3A, 3B and 3C, respectively), and will operate in the frequency range of 1-7 MHz as to provide good imaging resolution (higher ranges) and sufficient therapeutic focal power deposition (low-middle ranges) without in-path collateral damage.

Linear and annular phased arrays will provide three degrees of freedom and will allow electronic steering of the focal region in a three-dimensional domain without constraints. Sectioned annular arrays, on the other hand, will only allow electronic dynamic focusing on the propagation axis, in which case the transducer will be mechanically moved (up or down) and rotated on its long symmetry axis to provide complete sweeps of desired volumes. In this particular design, the loss in electronic steering freedom is compensated by a more efficient power transfer and focusing gain with greatly reduced side lobes.

Linear and spherical phased arrays are the preferred designs for external, transthoracic applications. In this approach, the strongly inhomogeneous nature of the intervening tissue between the transducer and the atrium requires maximum flexibility in the array phasing for accurate targeting and for minimizing phase aberrations that would significantly deteriorate the focal characteristics. Furthermore, because there are no major restrictions on the size of the HIFU system, a wide aperture and a large number of elements can be used to assure desired power deposition at deeper focal positions.

Conversely, given the limited circular dimension of the esophagus (circa 1.5 cm), small (e.g. 1 cm wide by 2-6 cm long) phased linear or phased sectioned annular array transducers will be the preferred embodiment. Because of the shape and orientation of the esophagus, the transducer may be larger in the dimension aligned with the esophageal axis. These transducers can be electronically steered in the plane of the image sector (as with linear phased array) or can be mechanically oscillated (as with an annular array). Both types will have the ability to electronically adjust the focal point of imaging and HIFU.

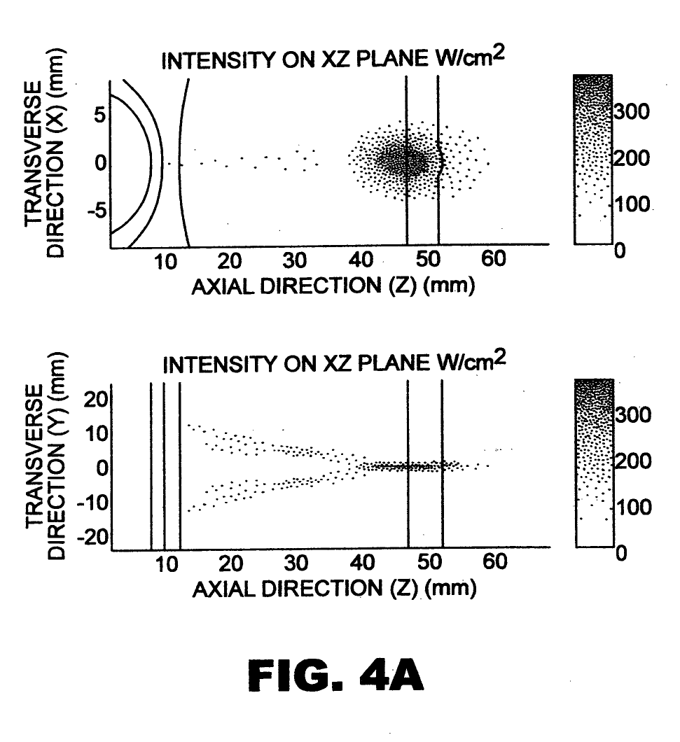

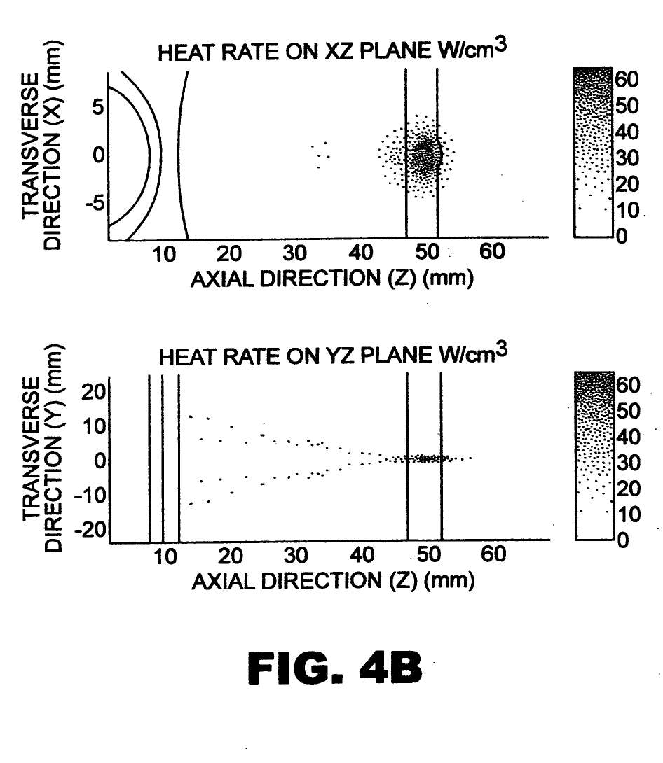

FIG. 4 shows the simulated field distributions of time averaged acoustic intensity (FIG. 4A) and heat rate (FIG. 4B) of a 20 element sectioned annular phased array, similar to that shown above in FIG. 3C, for transesophageal acoustic propagation in a model of the heart and focusing on the distal heart wall. For these simulations, the transducer aperture is assumed to be 4 cm along the axis of the esophagus and 1 cm in width. The HIFU system is located on the left inside the esophagus. The tissue layers correspond to esophageal wall, fluid, proximal heart wall, blood, distal heart wall, and fluid.

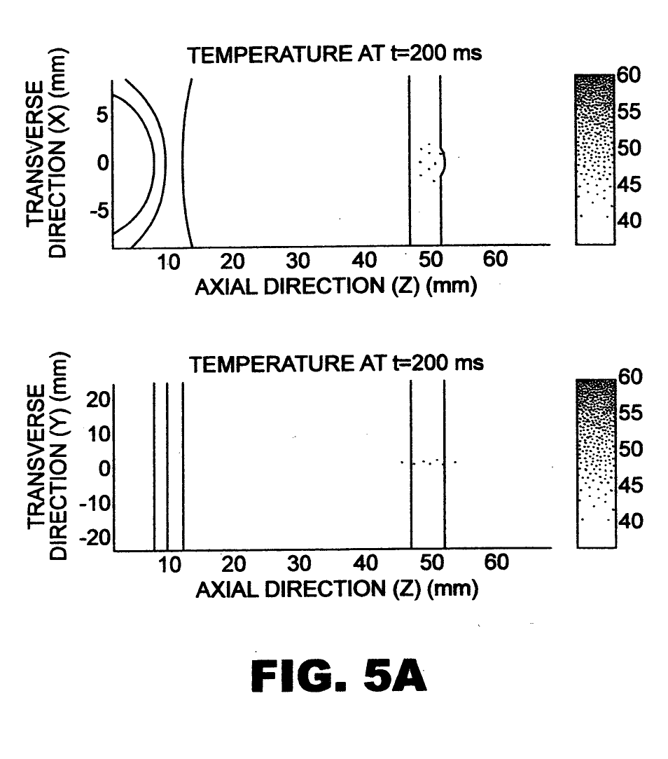

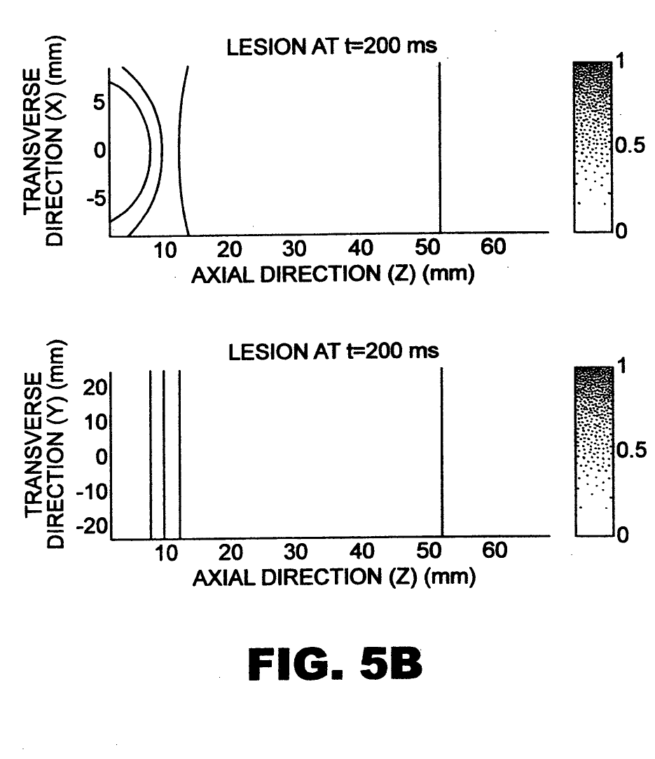

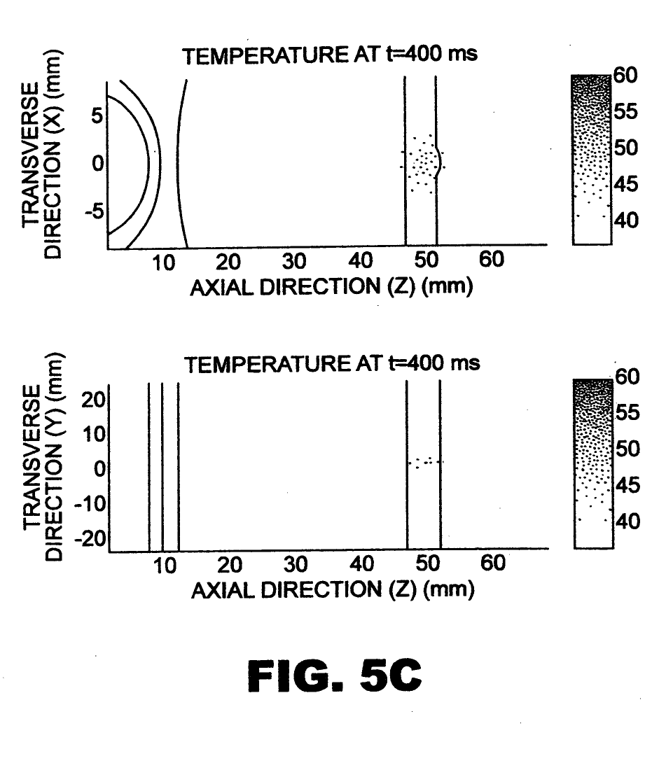

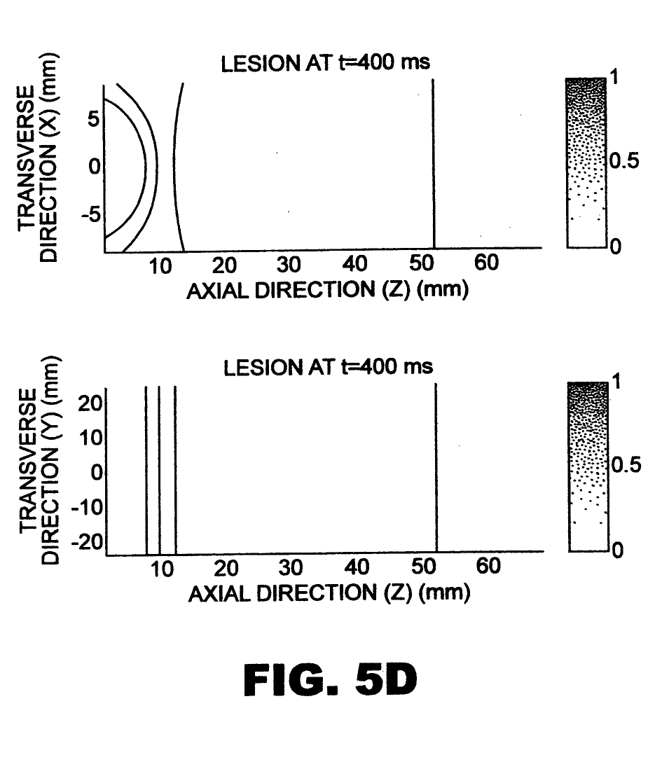

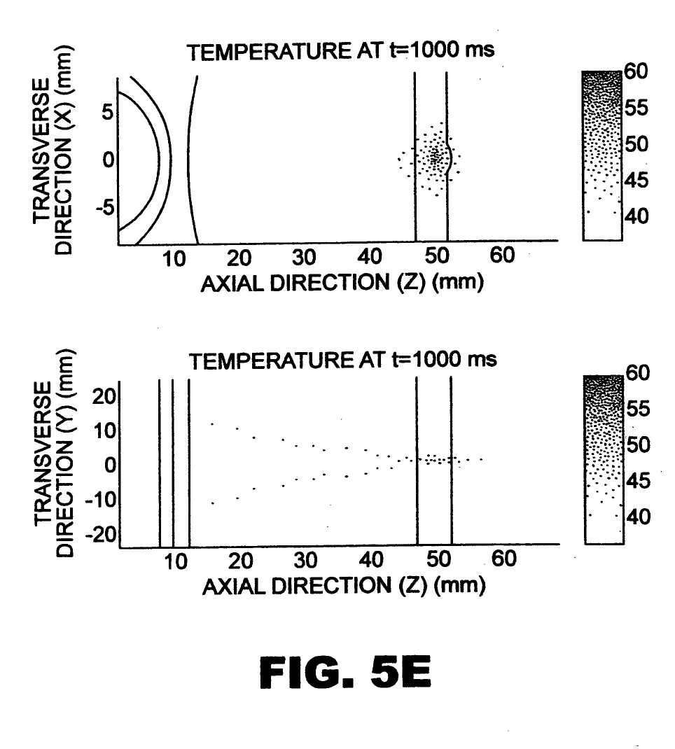

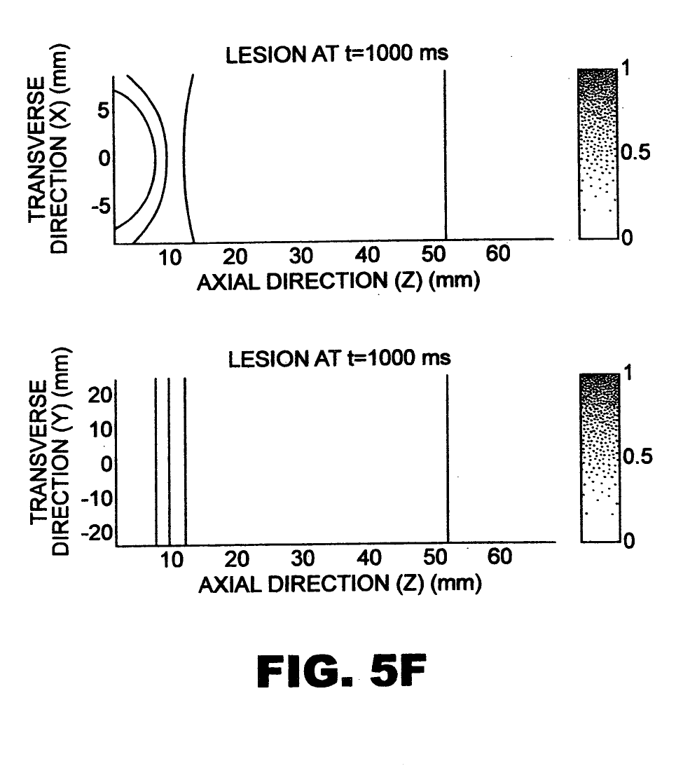

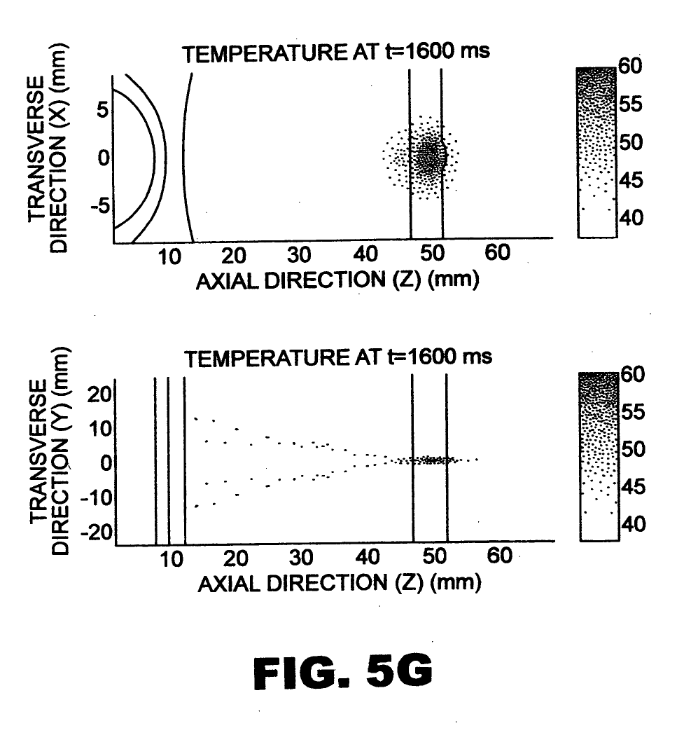

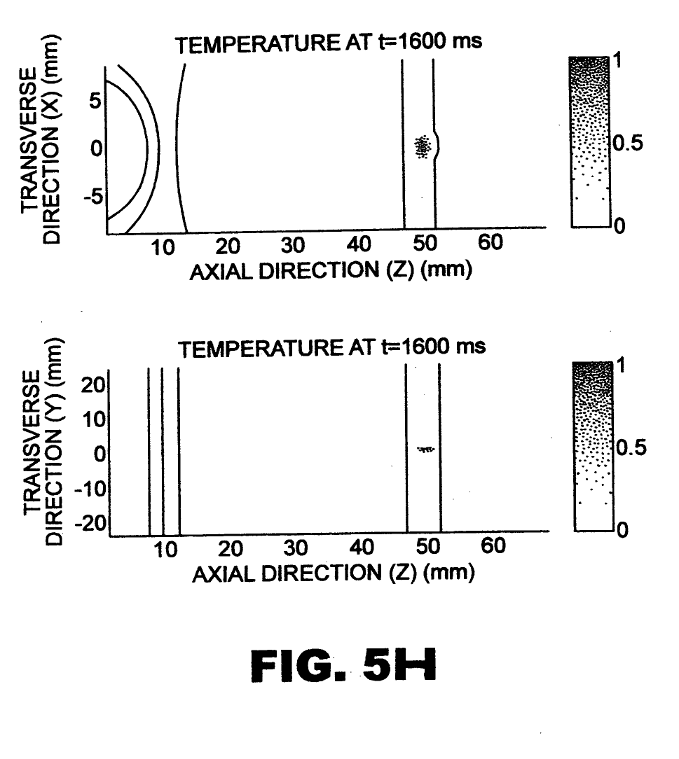

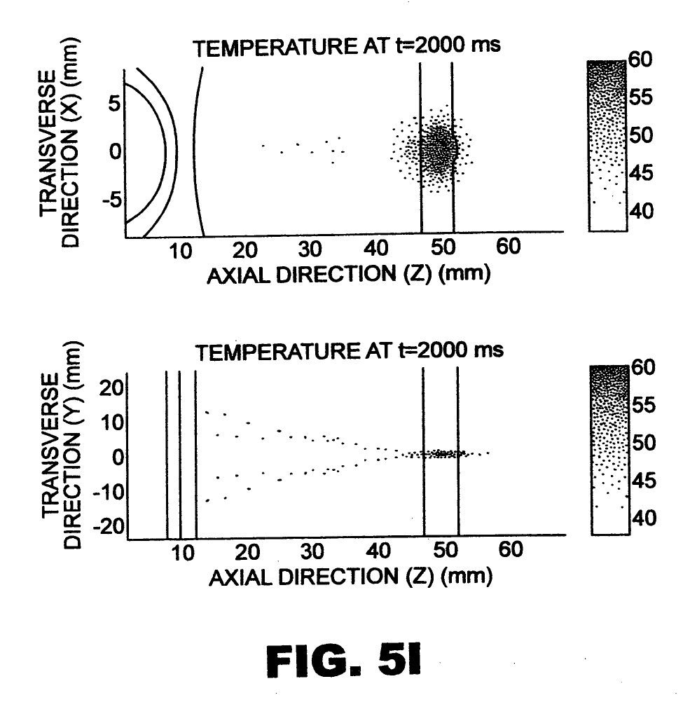

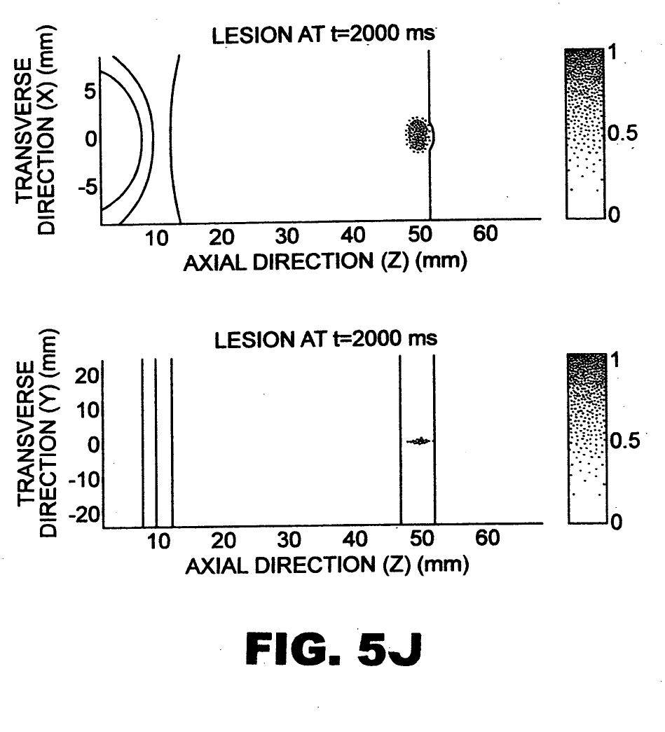

Based upon simulations of a proposed transducer design and under idealized acoustic propagation conditions (such as no moving blood, no scattering and no aberration generation), FIGS. 5A, 5C, 5E, 5G and 5I show temperature evolution at different time intervals while FIGS. 5B, 5D, 5F, 5H and 5J show respective lesion formation defined by the thermal dose criterion common to thermal therapy. Note that lesion formation is prevented until HIFU is applied for at least one second of continuous operation. For application in a beating heart with continuous flow of cooling blood, lesion formation will take several seconds. For illustration, see FIG. 1, where a lesion was formed in a beating pig heart in 10 seconds with the HIFU transducer placed on the epicardial surface. Direct observation has indicated that the desired lesion formation will take much longer than the time available during a single diastolic period. If multiple diastolic periods are incorporated into the therapy, the thermal cooling of tissue by the rapid bloodflow through the heart inhibits achieving the required thermal dose for the desired biologic effect.

Targeting only during periods when the heart is at rest results in the inability to achieve a thermal dose throughout the ROI to induce remodeling. Targeting the regions of the heart only while the heart is relatively stationary, such as during diastole results in the rapid conduction of heat away from the treated region by the blood, which remains near body temperature of 37 degrees Celsius, during the HIFU-off phase. One is prevented from using higher intensities to overcome this heat loss by the size of the transducers that would produce the HIFU lesion—at least for those contained within the esophagus. Increasing the power supplied to the transducers also is not an option because transducer heating will either damage the transducer element itself, or the esophagus.

For the invention described herein, targeting of the ROI in the diseased heart can be performed only dynamically with continuous or substantially continuous wave “CW” over a period of several heart cycles. Targeting the ROI with therapeutic ultrasound (HIFU) and the resulting thermal dose generation can be considered essentially continuous since any interruption for imaging would be very brief, on the order of only a few milliseconds, and can occur at any time throughout the heart cycle. HIFU therapy would continue through all cycles of the heart and therefore through all spatial positions of the ROI.

A principal difference between the approach outlined in the present invention and prior art as described in U.S. Pat. No. 5,817,021, is that the prior art recognizes the difficulties in treating the heart as a moving object. Accordingly, U.S. Pat. No. 5,817,021 teaches that it is better to use interrupted ultrasound and treat the heart only when it is in periods of rest, such as during diastole. This approach suffers from the problem that the heart is at rest for only relatively short periods of time (typically 0.3 to 0.5 sec during diastole for a normal heart at 75 beats per minute). Furthermore, in patients with cardiac disease the heart rate can be much faster and unstable, so that the rest period may be much shorter. The ventricular rate in patients with atrial fibrillation can range from 100 to 200 beats per minute (Kastor, Arrhythmias, Second Edition, 2000, page 52). Pharmaceutical approaches that slow the heart rate cannot slow the heart enough to obtain a satisfactorily long period of heart wall immobility and are not favored by clinicians as they often result in other complications.

Dynamic targeting can be accomplished in two ways. The first approach is where an electronic gate is placed around an echogenic structural landmark in the vicinity of the ROI (for example, the interface between the endocardial wall and blood) as determined from acquired B-mode images. The system (in imaging mode) will track the endocardium/blood interface echo within this gate as it moves axially and will generate a depth signal which will drive the HIFU transducer (in “CW” therapy mode) with the proper delays to move the focus accordingly to the heart motion.

The second approach of dynamic targeting involves the use of a micro ultrasonic device (transponder) mounted on an electrophysiology mapping catheter. The transponder will generate a source signal received by the therapy array and utilized with time-reversal algorithms to dynamically correct for phase aberrations resulting from multiple acoustic paths and compensate for the target motion. In this fashion, the focal region of the system will be able to continuously track the same target region as it moves. In this case, HIFU can be applied throughout the heart cycle, continually with brief inconsequential interruptions to acquire imaging frames, and lethal tissue damage can be obtained (see FIGS. 5H and 5J for example). FIGS. 5G and 5I show temperature evolution at time intervals of greater than one second, while FIGS. 5H and 5J show respective lesion (thermal dose criterion) formation due to continuous HIFU exposure for the model shown in FIGS. 4A and 4B. In this example, lesion formation is desired, and occurs exclusively into the endocardium due to the low absorption of both blood and external fluid. The applied HIFU therapy results in heating of the tissue to temperatures in excess of 65° C., and as shown in FIGS. 5H and 5J, with sufficient thermal dose to result in tissue necrosis.

The multi-element designs of the HIFU system provide flexibility in terms of focal spot dimensions. By properly choosing the individual phases and time delays of each element in the array, the focal dimensions and characteristics of the system can be manipulated from a high-power small, grain-of-rice-size focus, to a low-power large, navy-bean-size focal volume. For example, with an acoustic intensity on the order of 2 kW/cm² and a driving frequency of 2 MHz, tissue temperatures can be elevated to 100° C., from an ambient level of 37° C., within a few seconds. Modeling as illustrated in FIGS. 4, 5 and 6 accounts for nonlinear effects, tissue perfusion, temperature and frequency dependent absorption. Therefore, predicted temperatures can be as accurate to within a few degrees Celsius. With this level of control, it is possible to produce either sub-lethal or lethal tissue damage, with either a trans-esophageal or a trans-thoracic approach.

One of the strengths of HIFU over competing ablation technologies is the superior control that is available to the user, and this control takes many forms. For example, because the focal volume of the therapy transducer is normally small, one has relatively precise control over the spatial extend of the tissue lesion that is produced. Finally, because the duration of the applied HIFU can be controlled so precisely (to within a few acoustic cycles at 2 MHz), local tissue temperatures can be controlled to within a few degrees Celsius. This temperature control allows one to selectively treat different tissue types. For example, muscle tissue can be necrosed but the vasculature remains intact, due to the cooling effect of blood within the vessels. In addition, connective tissues are more capable of withstanding elevated temperatures than muscle cells, and thus, with proper control of the local tissue temperature, myocardial tissues can be necrosed without damage to the surrounding matrix of connective tissues. Nerve tissue are the least capable of withstanding elevated temperatures, providing the opportunity of selectively destroying nerve budding surrounding an infarct area.

The present invention provides patient benefits which include:
1. a unique, durable non-invasive or minimally invasive therapeutic approach to the beating heart for the treatment of heart tissues damaged, destroyed and scarred by myocardial infarction.
2. Infarct areas of the heart (areas of necrosis resulting from a sudden insufficiency of the blood supply), such as tissue in the heart wall that has been scarred as a result of a heart attack, may be treated with HIFU. HIFU thermal therapy may be used to induce sub-lethal heating (described above) to induce or cause angiogenesis (new blood vessels forming), the formation of myocytes (cardiac muscle cells), and may disrupt or destroy nerve budding while preserving myocytes. The resultant is a remodeling tissue to functioning tissue, which may also include a simultaneous reduction or elimination of nerve budding stimulated by the infarct which, if left to develop into new nerves, may give rise to ventricular arrhythmia including fibrillation.
3. the ability to repeat the therapeutic ultrasound cardiac myocardium therapy procedure indefinitely with only minor morbidity.

While the invention has been described with reference to preferred embodiments it is to be understood that the invention is not limited to the particulars thereof. The present invention is intended to include modifications which would be apparent to those skilled in the art to which the subject matter pertains without deviating from the spirit and scope of the appended claims.

1. A method for reducing the debilitating effects of myocardial infarctions in a heart, said method comprising:
targeting a region of interest of the heart by diagnostic imaging;
emitting without timing to the heart cycle or position, and in a continual manner, therapeutic ultrasound energy from an ultrasound radiating surface placed on the skin or in the esophagus;
focusing the emitted therapeutic ultrasound energy on the region of interest throughout the heart cycle; and,
producing sub-lethal or partially lethal tissue or cellular damage in the region of interest thereby inducing at least one of regeneration of myocardial cells and angiogenesis in the region of interest.

2. The method of claim 1 wherein said targeting is carried out with diagnostic ultrasound.

3. The method of claim 1 wherein said targeting is carried out with Magnetic Resonance Imaging.

4. The method of claim 1 wherein the region of interest comprises a ventricular wall of the heart.

5. The method of claim 1 in which the ultrasound radiating surface is located in the esophagus.

6. The method of claim 1 in which the ultrasound radiating surface is located on the skin and the energy is delivered transthoracically.

7. The method of claim 6 wherein the energy is delivered intercostally or subcostally.

8. The method of claim 2 in which pulse echo signals from the diagnostic array is used to deliver the emitted therapeutic ultrasound energy in phase with the heart motion thereby delivering ultrasound energy in a continual manner without respect to the timing or phase of the heart cycle, interrupted only briefly to acquire imaging frames.

9. The method of claim 1 in which the emitted ultrasound energy produces sub-lethal tissue damage in a region of the infarction or other regions of interest.

10. The method of claim 1 wherein said therapeutic ultrasound comprises high intensity focused ultrasound (HIFU) emitted in a substantially continuous manner without respect to timing or phase of the cardiac cycle.

11. The method of claim 2 wherein said targeting further comprises:
placing an ultrasonic device at the region of interest, said device generating a signal which identifies the infarction via the diagnostic imaging and provides a focus location for the therapeutic ultrasound, and wherein the device signal is also received by the imaging transducer and electronics which provide phase aberration correction feedback data to the therapeutic ultrasound system to accurately generate the therapeutic ultrasound focus and to overcome diffraction limits by expanding the effective aperture of the therapeutic ultrasound transducer.

12. A method for providing for non-invasive or minimally invasive treatment of myocardial infarction utilizing therapeutic ultrasound, emitted without respect to the timing or phase of the cardiac cycle, said method comprising:
inducing at least one of injury to cardiac cells, phase transitions, changes in the shape of cell proteins, and structural protein remodeling in a defined volume, whereby tissues regenerate over time in a manner which reduces the volume of the infarction and increases cardiac output.

13. The method of claim 12 wherein said inducing is effected with High Intensity Focused Ultrasound.
